SEQUENCE LISTING (1 sequence)

SEQ ID NO: 1 - protein, 189 aa, Artificial sequence
  Synthetic construct
MTEYKLVVVGAGGVGKNALTIQLIQNHFVDEYDPTIEDSYRKQVVIDGETCLLDILDTAGLEEYSAMRDQSMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

(19) United States
(12) Patent Application Publication
(10) Pub. No.: US 2008/0199505 A1
(43) Pub. Date: Aug. 21, 2008

(54) Local Gene Therapy with an Eluting Stent for Vascular Injury

(76) Inventor: Shu Chien, La Jolla, CA (US)

Correspondence Address: DLA PIPER US LLP, 4365 EXECUTIVE DRIVE, SUITE 1100, SAN DIEGO, CA, 92121-2133, US

(21) Appl. No.: 11/883,189

(22) PCT Filed: Feb. 3, 2006
(86) PCT No.: PCT/US2006/003963

§ 371 Date: Mar. 20, 2008

Related U.S. Application Data
(60) Provisional application No. 60/650,069, filed on Feb. 4, 2005.

(51) Int. Cl. A61F 2/00; A61K 35/76; A61P 9/00

(52) U.S. Cl. 424/423; 424/93.2

(57) ABSTRACT

The present invention relates to a method for preventing or treating stenosis or restenosis in subjects by placement of a polymerized support delivering a recombinant adeno or adeno-associated vector with a mutant Ras nucleic acid encoding a mutant Ras protein which inhibits Ras-mediated phosphorylation, and blocking the Ras signal transduction pathway in vascular smooth muscle cells (VSMCs).

### RELATED APPLICATION

This application claims priority under 35 U.S.C. §119(e) to U.S. Provisional Application 60/650,069 filed Feb. 4, 2005, herein incorporated by reference in its entirety.

### FIELD OF THE INVENTION

The present invention relates to a methods for preventing or treating disorders associated with vascular injury caused by mechanical stimuli including restenosis, atherosclerosis and reperfusion injury. In particular, the invention relates to methods for delivery of biological therapeutic agents capable of inhibiting the Ras signal transduction pathway and proliferation of vascular cells at the injury site.

### BACKGROUND INFORMATION

A major process through which extracellular stimuli can be transmitted into cells involves the membrane-associated p21.sup.ras and its downstream cytoplasmic kinase pathways, especially the members in the mitogen-activated protein kinases (MAPK) family. p21.sup.ras is a small GTPase molecule that plays a key role in the signal transduction pathways of cellular responses to stimuli by mitogens, cytokines, environmental stresses, and UV irradiation. p21.sup.ras cycles between an active GTP-bound state and an inactive GDP-bound state, thereby functioning as a molecular switch in response to extracellular stimuli in the control of normal and transformed cell growth. Activated p21.sup.ras triggers two protein kinases, Raf-1 and MAPK kinase (or MEK kinase, MEKK) which activate the downstream MAPKs, including c-Jun NH2-terminal kinases (JNK) and extracellular signal-regulated kinases ERK (11,32). Raf-1 activates ERK but not JNK, whereas MEKK mediates preferentially JNK over ERK (32,48). In different types of cells in response to UV irradiation, Ha-Ras expression, and osmotic shock, JNK kinase (JNKK) activates JNK by phosphorylating the Thr-Pro-Tyr phosphorylation sites, and the activated JNIK binds to c-Jun to specifically phosphorylate the −63 and −73 amino acids at the N-terminal (10, 29). In response to Ha-Ras expression, serum growth factor, or phorbol ester TPA stimulation, MEK activates ERK which in turn phosphorylates the transcription factor p62 ternary complex factor (p62TCF), leading to the activation of c-Fos (5, 18, 30, 37). In R.EF-52 fibroblasts, the activation of AP-I/TRE by these stimuli is mediated through ERK (16). It is not known where and how mechanical stimuli are transduced to biochemical signals. p21.sup.ras is a membrane-associated protein and its activation of the downstream Raf-1 and MEKK is through direct interactions on the membrane. Wang et al. (47 suggest that the integrins on the basal membrane constitute a mechano-receptor and that actin stress fibers are necessary to transmit the applied forces. Similarly, Davies et al. (8) suggest that focal adhesion complex at the abluminal endothelial membrane are mechanically responsive elements coupled to the cytoskeleton.

Ras can activate both ERK and JNK pathways. See Kyriakis, et al., “The Stress-Activated Protein Kinase Subfamily of c-Jun Kinases,” Nature, 369:156-160, 1994; and Marshall, C. J., “Specificity of Receptor Tyrosine Kinase Signaling: Transient Versus Sustained Extracellular Signal-Regulated Kinase Activation,” Cell, 80:179-185, 1995. The signaling in response to growth factors such as epidermal growth factor (EGF) and nerve growth factor (NGF) is mediated through both the Ras/ERK and Ras/JNK pathways in PCI2, MRCS, and HeLa cells. See Marshall (1995), supra; and Minden, et al., “Differential Activation of ERK and JNK Mitogen-Activated Protein Kinases by Raf-1 and MEKK,” Science, 266:1719-1723, 1994.

In contrast, inflammation—related cytokines (e.g., TNF and IL-1), environment stresses (e.g., osmotic pressure), and UV irradiation selectively activate the Ras/JNK, but not the ERK, pathway. See Galcheva-Gargova, et al, “an Osmosensing Signal Transduction Pathway in Mammalian Cells,” Science, 265:806-808, 1994; Hibi, et al., “Identification of an Oncoprotein and UV Responsive Protein Kinase That Binds and Potentiates the c-Jun Activation Domain,” Genes Dev., 7:2135-2148, 1993; Kuchan, et al., “Role of G Proteins in Shear Stress-Mediated Nitric Oxide Production by Endothelial Cells,” Am. J. Physiol., 267(3 Pt 1):C753-758, 1994; Marshall (1995), supra; Shyy, et al., “The cis-Acting Phorbol Ester “12-O-Tetradecanoylphorbol 13-Acetate”-Responsive Element is Involved in Shear Stress-Induced Monocyte Chemotactic Protein 1 Gene Expression’; and Stokoe, et al., “Activation of Rafas a Result of Recruitment of the Plasma Membrane,” Science, 264:1463-1467, 199. Mechanical shearing, which results from the flow of blood or other fluids, is a form of force borne by vascular ECs and many other cell types, such as the osteoblasts, under physiological conditions.

The application of such physiological forces on static cells cultured in the flow chambers provides a sudden change of hemodynamic environment. This in vitro system mimics the pathophysiological changes during reperfusion after flow stoppage. Fluid shearing of vascular EC caused the activation of JNK by more than 10-fold and the activation of ERK by a much lesser magnitude (1.8 fold) and shorter duration. Morooka et al. demonstrated that reperfusion of ischemic kidney induced a rapid activation of JNK. See Minden, et al., “Selective Activation of the JNK Signaling Cascade and c-Jun Transcriptional Activity by the Small GTPases Rac and Cdc-42Hs,” Cell, 81:1147-1157, 1995. Bogoyevitch et al. reported that reperfusion of rat heart induced a 10- to 50-fold activation of JNK, but not ERK. See Bogoyevitch, et al., “Ischemia and Reperfusion Activates Jun N-Terminal Protein Kinase (JNKs) in the Adult Rat Ventricular Myocardium,” Circulation, 92:1-571, 1995.

Stents containing drugs inhibiting proliferation, inflammation and thrombosis have been used for local drug delivery treatment of restenosis in preclinical and clinical studies. It has been demonstrated that adenovirus-mediated RasN17 significantly inhibits neointima (the new layer of endothelial cells and the associated matrix) on the intimal surface of blood vessel graft or vascular prosthesis), formation in pig coronary arteries. See U.S. Pat. No. 6,335,010 to Chien et al., filed Jun. 30, 1997, which is hereby incorporated by reference in its entirety.

Coated stent gene therapy inhibiting a Ras signal transduction pathway has not yet been described. The coated stent gene therapy method may result in locally transformed tissues with a longer lasting or more permanent inhibitory effect on cell proliferation, inflammation and thrombosis than drug-coated stents.

### SUMMARY OF THE INVENTION

The present invention relates to methods of gene therapy delivery for human vascular disorders which are capable of localizing and enhancing the expression of the gene therapy vectors. The present invention provides delivery methods utilizing a stent coated with polymers containing a therapeutic agent, for example, RasN17 or other signaling impeding proteins, at the site of vascular injury, for example, delivery to prevent neointima formation.

The methods of the present invention combines several of the following characteristics: (1) the molecular mechanism-based protection resulting from blocking ras signaling pathway in proliferating and migrating vascular cells; (2) target site specific delivery; and (3) high concentrations of the therapeutic product at the delivery site.

### DETAILED DESCRIPTION OF THE INVENTION

The present invention may be understood more readily by reference to the following detailed description of specific embodiments and the Examples included therein.

Stenosis is narrowing of a duct or canal, for example, a blood vessel. Restenosis is narrowing of a structure following the removal or reduction of a previous narrowing, for example, following balloon angioplasty of a blood vessel. Restenois is major disadvantage of angioplasty and bypass. Generally, doctors in the clinic use stents are to reduce restenosis rate. However, the problem of in-stent restenosis is becoming the major limitation for the widespread use of this device. Recent studies have shown that certain cellular responses occur due to mechanical stimuli. For example, vascular smooth muscle cells (VMSC) have been shown to proliferate after inserting the stent is inserted. This is one form of restenosis.

Several mutant proteins (signaling incompetent versions of Ras, Src, MEKK, and JNK) can act in a dominant negative manner to block the ras signal transduction pathway. Previous experiments have shown that using adenovirus-mediated dominant negative mutant RasN17 led to a 56% decrease in neointima formation and a 75% increase in lumen size in pig coronary arteries.

SEQ ID NO:1 is the full-length amino acid sequence of ras, positions 1-190.

```
(SEQ ID NO: 1)
Met Thr Glu Tyr Lys Leu Val Val Val Gly Ala Gly Gly Val Gly Lys
  1 5 10 15
Asn Ala Leu Thr Ile Gln Leu Ile Gln Asn His Phe Val Asp Glu Tyr
                        20 25 30
Asp Pro Thr Ile Glu Asp Ser Tyr Arg Lys Gln Val Val Ile Asp Gly
                35 40 45
Glu Thr Cys Leu Leu Asp Ile Leu Asp Thr Ala Gly Leu Glu Glu Tyr
        50 55 60
Ser Ala Met Arg Asp Gln Ser Met Arg Thr Gly Glu Gly Phe Leu Cys
65 70 75 80
Val Phe Ala Ile Asn Asn Thr Lys Ser Phe Glu Asp Ile His Gln Tyr
                                85 90 95
Arg Glu Gln Ile Lys Arg Val Lys Asp Ser Asp Asp Val Pro Met Val
                        100 105 110
Leu Val Gly Asn Lys Cys Asp Leu Ala Ala Arg Thr Val Glu Ser Arg
                115 120 125
Gln Ala Gln Asp Leu Ala Arg Ser Tyr Gly Ile Pro Tyr Ile Glu Thr
        130 135 140
Ser Ala Lys Thr Arg Gln Gly Val Glu Asp Ala Phe Tyr Thr Leu Val
145 150 155 160
Arg Glu Ile Arg Gln His Lys Leu Arg Lys Leu Asn Pro Pro Asp Glu
                                165 170 175
Ser Gly Pro Gly Cys Met Ser Cys Lys Cys Val Leu Ser
```

The present invention relates to a gene of interest, or a nucleic acid molecule, which can be any nucleic acid molecule of interest, including DNA and/or RNA, and can be identified, for example, based on the presence (or absence) of a mutation or a polymorphism (e.g., a single nucleotide polymorphism (SNP). The gene of interest also can be an oncogene or a tumor suppressor gene, a bacterial nucleic acid molecule (e.g., to determine whether an individual has an infection, or whether an infecting bacterium has a gene that may be susceptible (or resistant) to a particular antibiotic), or a viral nucleic acid molecule (e.g., to identify or confirm the presence of a viral infection), or can be an mRNA molecule (e.g., to detect expression, or lack thereof, of a particular gene, such as in response to an agent being tested to determine whether it can effect regulation of the gene). A gene of interest also can be, for example, a cDNA molecule, a small interfering RNA molecule (siRNA, RNAi), and/or an aptamer.

DNA encoding the peptides (i.e., ras, MEKK, JNKK, JNK, ERK, p21, p53, etc.,) of the invention can be inserted into an expression vector. The term “expression vector” refers to a genetic construct such as a plasmid, virus or other vehicle known in the art that can be engineered to contain a nucleic acid encoding a polypeptide of the invention. Such expression vectors are preferably plasmids that contain a promoter sequence that facilitates transcription of the inserted genetic sequence in a host cell. The expression vector typically contains an origin of replication and a promoter, as well as polynucleotides that allow phenotypic selection of the transformed cells (e.g., an antibiotic-resistant polynucleotide). Various promoters, including inducible and constitutive promoters, can be utilized in the invention. Typically, the expression vector contains a replicon site and control sequences that are derived from a species compatible with the host cell.

The present invention is based on adeno viral vectors, as previously described in U.S. Pat. No. 6,335,010B1 to Chien et al. Unlike retroviruses which integrate into the host genome, adenoviruses persist extrachromosomally thus minimizing the risks associated with insertional mutagenesis (Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; and Rich et al. (1993) Human Gene Therapy 4:461-476).

The present invention also provides for adeno-associated virus (AAV) vector system, which have been developed for polynucleotide delivery or delivery of gene(s) of interest. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158; 97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Additional viral vectors useful for delivering the polynucleotides encoding polypeptides of the present invention by gene transfer include those derived from the pox family of viruses, such as vaccinia virus and avian poxvirus. By way of example, vaccinia virus recombinants expressing the novel molecules can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK.sup.(−) recombinant can be selected by culturing the cells in the presence of 5-bromodeoxyuridine and picking viral plaques resistant thereto.

A vaccinia-based infection/transfection system can be conveniently used to provide for inducible, transient expression or coexpression of one or more polypeptides described herein in host cells of an organism. In this particular system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide or polynucleotides of interest, driven by a T7 promoter. The polymerase expressed in the cytoplasm from the vaccina virus recombinant transcribes the transfected DNA into RNA which is then translated into polypeptide by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al. Proc. Natl. Acad. Sci. USA (1986) 83:8122-8126.

Alternatively, avipoxviruse vectors, such as the fowlpox and canarypox viruses, can also be used to deliver the coding sequences of interest. Recombinant avipox viruses, expressing immunogens from mammalian pathogens, are known to confer protective immunity when administered to non-avian species. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Still, any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al. J. Biol. Chem. (1993) 268:6866-6869 and Wagner et al. Proc. Natl. Acad. Sci. USA (1992) 89:6099-6103, can also be used for gene delivery under the invention.

Additional illustrative information on these and other known viral-based delivery systems can be found, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993.

Still another aspect of the present invention provides delivery of the therapeutic agent(s) effectively at the site of injury of a blood vessel or other tissues via a therapeutic agent-containing polymer-coated stent or other similar semi-solid or solid support. An effective amount of therapeutic agent is achieved for a designed period with a low systemic side effects. Further, the present invention provides for a complex of DNA, protein, or lipids, formed in such a way to effectively transfect vascular smooth muscle cells at the injury site.

Although the present process has been described with reference to specific details of certain embodiments thereof in the above examples, it will be understood that modifications and variations are encompassed within the spirit and scope of the invention. Accordingly, the invention is limited only by the following claims.

Upon entry of the present amendment, the claims will stand as follows in the present application:

1. A method of inhibiting the proliferation of Ras signal transduction pathway-dependent vascular smooth muscle cells (VSMCs) at a vascular injured site, the method comprising: delivering to VSMCs at the vascular injured site by placement of a support into a subject, the support having a polymer coating further comprising a mutant Ras nucleic acid encoding a mutant Ras protein, wherein the nucleic acid is delivered in a replication defective adeno or adeno-associated viral vector, thereby blocking a Ras signal transduction pathway.

2. A method of inhibiting the occurrence of Ras signal transduction pathway-dependent restenosis resulting from an angioplasty procedure, the method comprising: delivering by placement of a support in close proximity to the angioplasty site a mutant Ras nucleic acid encoding a mutant Ras protein, wherein the nucleic acid is delivered in a replication defective adeno or adeno-associated viral vector, thereby inhibiting the occurrence of Ras signal transduction pathway-dependent restenosis resulting from an angioplasty procedure.

3. The method of claim 1 wherein the support is a semi-solid polymeric support.

4. The method of claim 1 wherein the support is a stent.

5. The method of claim 3 wherein the stent is further comprised of a polymer coating.

6. The method of claim 1, wherein the ras gene is delivered in a replication defective adeno virus vector.

7. The method of claim 1, wherein the ras gene is delivered in a replication defective adeno-associated virus vector.

8. The method of claim 1, wherein the mutant Ras protein contains amino acid sequence SEQ ID NO: 1 wherein residue 17 is glycine or lysine.

9. The method of claim 1, wherein the mutant Ras protein inhibits Ras-mediated phosphorylation.

10. The method of claim 9, wherein the mutant Ras protein inhibits the proliferation of Ras signal transduction pathway-dependent in VSMCs.

11. The method of claim 1, wherein the mutant Ras protein contains amino acid residues 1 to 25 of the amino acid sequence SEQ ID NO: 1, wherein residue 17 is glycine or lysine.

12. An article of manufacture, comprising a stent and a coating disposed on the stent, wherein the coating comprises a polymer and a therapeutic composition, the therapeutic composition including:
(a) a mutant Ras nucleic acid encoding a mutant Ras protein; and
(b) transfection viral vector.

13. The article of manufacture of claim 12, wherein the mutant Ras nucleic acid is an Ras N17 positive negative mutant.

14. The article of manufacture of claim 13, wherein the Ras N17 positive negative mutant is a dominant negative mutant.

15. The article of manufacture of claim 12, wherein the therapeutic composition further includes an expression promoter.

16. The method of claim 2 wherein the support is a semi-solid polymeric support.

17. The method of claim 2 wherein the support is a stent.

18. The method of claim 2, wherein the ras gene is delivered in a replication defective adeno virus vector.

19. The method of claim 2, wherein the ras gene is delivered in a replication defective adeno-associated virus vector.

20. The method of claim 2, wherein the mutant Ras protein contains amino acid sequence SEQ ID NO: 1 wherein residue 17 is glycine or lysine.

21. The method of claim 2, wherein the mutant Ras protein inhibits Ras-mediated phosphorylation.

22. The method of claim 2, wherein the mutant Ras protein contains amino acid residues 1 to 25 of the amino acid sequence SEQ ID NO: 1, wherein residue 17 is glycine or lysine.
